SEQUENCE LISTING (4 sequences)

SEQ ID NO: 1 - protein, 10 aa, Homo sapiens
  MISC_FEATURE (1)..(1): Alternative residue Ala, Pro
XXXXXXAXXS

SEQ ID NO: 2 - protein, 10 aa, Homo sapiens
  MISC_FEATURE (6)..(6): Xaa is unknown
TKAARXSAPX

SEQ ID NO: 3 - protein, 10 aa, Bos sp.
ARTKQTARKS

SEQ ID NO: 4 - protein, 10 aa, Bos sp.
TKAARKSAPA

(19) United States
(12) Patent Application Publication
(10) Pub. No.: US 2006/0233807 A1
(43) Pub. Date: Oct. 19, 2006

(54) Novel therapies and methods of screening for therapeutic compounds

(76) Inventor: Catharina Svanborg, Lund (SE)

Correspondence Address: NIXON & VANDERHYE, PC, 901 NORTH GLEBE ROAD, 11TH FLOOR, ARLINGTON, VA, 22203, US

(21) Appl. No.: 10/515,576

(22) PCT Filed: May 22, 2003
(86) PCT No.: PCT/IB03/02587

§ 371 Date: Jun. 21, 2005

(30) Foreign Application Priority Data
May 22, 2002 (GB) 0211658.0

(51) Int. Cl. A61K 48/00; A61K 39/395

(52) U.S. Cl. 424/155.1; 514/44

(57) ABSTRACT

A method for killing cells, said method comprising introducing into the nucleus of the cell, a moiety (other than HAMLET) which inhibits histone activity. This method will be useful for example in the treatment of cancer for antibacterial activity. Histones can be used as a basis of screening methods to select therapeutic compounds, and these are also described and claimed.

The present invention relates to methods for killing cells, as well as to the use of these methods in therapy, for example in the treatment of tumours or for antibacterial applications. The invention further provides methods for screening for compounds or reagents which would be useful in these therapeutic methods, as well as to novel reagents useful in this way.

HAMLET (Human α-lactalbumin made lethal to tumor cells) is a molecular complex that induces cell death in tumor cells. Indeed, the effect is selective for tumor cells and some immature cells and healthy, differentiated cells do not undergo cell death in response to HAMLET. This selectivity implies that HAMLET reaches unique targets in tumor cells, but not in resistant cells.

As used herein, the term “HAMLET” refers to a biologically active complex of α-lactalbumin (which may or may not be human in origin), which is either obtainable by isolation from casein fractions of milk which have been precipitated at pH 4.6, by a combination of anion exchange and gel chromatography as described for example in EP-A-0776214, or by subjecting α-lactalbumin to ion exchange chromatography in the presence of a cofactor from human milk casein, characterized as C18:1 fatty acid as described in WO99/26979.

The cellular targets for HAMLET have been examined by a combination of confocal microscopy and subcellular fractionation [H\dot{a}kansson et al., 1999 Exp Cell Res. 246, 451-60]. HAMLET binds to the cell surface, and enters the cytoplasm where it interacts with and activates mitochondria. Finally, the protein enters the cell nuclei, where it accumulates.

The applicants have found that resistant and sensitive cells bind HAMLET to their surface with similar efficiency, suggesting that this is not the discriminating event. The nuclear accumulation, in contrast, occurs only in dying cells, suggesting that this step distinguishes sensitive from resistant cells. By confocal microscopy, the nuclear accumulation appeared irreversible, suggesting the presence of nuclear targets that bind and retain HAMLET in the nuclear compartment.

The applicants have conducted a closer examination of the intranuclear distribution of HAMLET and have found that the protein preferentially localized to areas corresponding to the nucleoli, implying that HAMLET interacts with molecules involved in the regulation of chromatin structure.

As a result of further study, the nuclear targets for HAMLET were identified. Surprisingly it was found that HAMLET interacts with specific histone proteins and with nucleosomes. It appears therefore that this interaction may be the event that irreversibly locks the cells into the death pathway.

Tumours are often insensitive to stimuli that induce cell death in healthy cells, resulting in increased longevity. They proliferate in an uncontrolled manner and fail to respect tissue boundaries. The ability of HAMLET to selectively induce cell death in tumor cells is quite unusual. It kills a wide range of tumor cells including lymphomas and carcinomas of human and other origin. Understanding of the cellular targets for HAMLET may thus be useful for the understanding of cell death programs that remain functional in tumor cells. The nuclear effects of HAMLET demonstrate a new mechanism for DNA injury and cell death. This new mode of action gives rise to new therapeutic and screening possibilities.

According to the present invention there is provided a method for killing cells, said method comprising introducing into the nucleus of the cell, a moiety (other than HAMLET) which inhibits histone activity.

The core histones are evolutionarily conserved proteins that serve to maintain DNA integrity and function. They are highly charged, basic proteins with a low degree of primary structure homology, but share a tertiary structure motif, the histone fold [Arents et al., 1991 Proc. Natl. Acad. Sci. USA, 88, 10148-52] that is responsible for the major DNA binding. The nucleosome consists of a core histone octamer wrapped with approximately 146 bp of DNA in 1.65 turns of a left-handed superhelix (Arents et al, 1993 Proc. Natl. Acad. Sci. USA, 90, 10489-93]. Adjacent nucleosomes are connected with linker DNA and the linker histone H1. The core histone octamer consists of one (H3-H4)₂ tetramer, and two H2A-H2B dimers positioned on each side of the tetramer. The N-terminal histone tails regulate the higher order chromatin structure and may undergo posttranslational modifications such as acetylation, phosphorylation and methylation [Strahl, 2000 Nature. 403, 41-5].

In order to inhibit histone activity, the moiety will generally bind to the histone molecule, and prevent nucleosome formation in the case of free histones, or in chromatin formation in the case of ready formed nucleosomes.

In particular, the moiety should bind H2, H3 and/or H4 histones, preferably the H3 and/or H4 histones. In one embodiment the moiety should bind the H4 histone. In another embodiment, it should bind the H3 histone. Suitably the moiety binds free histones which sufficiently high affinity to prevent their being used in nucleosome assembly. This means that the moiety will mimic the binding of HAMLET in cell nuclei.

As described in more detail hereinafter, the applicants have examined the molecular basis for the nuclear accumulation of HAMLET in tumor cells, and identified histones as nuclear targets.

HAMLET was shown to interact with histones in intact nucleosomes, and at low concentrations, HAMLET triggered the assembly of free histones onto DNA fragments at physiological ionic strength. As HAMLET does not bind DNA, this effect must be mediated through the histones.

Proteins interacting with HAMLET were isolated from nuclear fractions and identified as histones, and the specificity was confirmed using purified histones. HAMLET showed high affinity for histones H3, intermediate affinity for H4, lower affinity for H2A and H2B and no affinity for H1. HAMLET was subsequently shown to interact with native histones in preformed nucleosomes and to affect nucleosome assembly. It seems likely that the nuclear accumulation of HAMLET is caused by this affinity for histones and nucleosomes and this suggests that this interaction leads to disruption of the chromatin structure.

In order to mimic the activity of HAMLET, the moiety used in the method of the invention should interfere with this activity. Thus it may comprise a moiety which specifically binds to the histone, such as an antibody or a binding fragment thereof, in such a way that the chromatin assembly or remodeling activity of the cell is inhibited. Alternatively, it may be a moiety that acts at the DNA or RNA level to inhibit expression of functional protein. Such moieties may comprise for example sense or antisense RNA constructs as would be understood in the art.

The moiety should be applied to the cell in a manner in which it will penetrate the cell and enter the nucleus. Where this does not occur naturally (as with HAMLET), the moiety may be attached to a carrier protein or peptide, which is able to translocate the moiety into the cell. Examples of translocation factors include the tat protein from HIV or the herpes simplex virus type I tegument protein VP22 or a functional fragment or homologue thereof (see for example WO 98/32866, as well as the homeodomain of Antennapedia as described in WO99/11809.

Alternatively, where the moiety is a protein or peptide, such as an antibody, intracellular expression of the protein or peptide may be carried out. In this case the cells are contacted with a vector such as a plasmid or virus vector which is able to enter the cell, which has contains a nucleic acid sequence which encodes the moiety, and expresses that sequence in the cell.

Particular examples of vectors may include virus vectors which have a nuclear targeting mode such as adenovirus vectors.

Alternatively, the moiety may further comprise a targeting entity that specifically target the nucleii within the cell, to ensure that the inhibition of histones occurs in the most efficient manner to cause cell death. For example, one might include a nuclear targeting signal peptide (NTS) derived from a virus such as an alphavirus. In general, the nuclear targeting signals are located in a lysine rich area of the genome and will comprise a region which has at least 3 and generally 4 adjacent lysines such as described by Chelsky et al., 1989, Mol+Cell Biology, 9, p 2487-2492.

In order to further mimic the activity of HAMLET, the moiety may further comprise a targeting reagent which is specific for tumour or undifferentiated cells, such as a tumour specific antibody or a binding fragment thereof.

A further aspect of the invention comprises a method of screening for moieties which may kill cells, said method comprises contacting a moiety under test with a histone, and detecting an interaction therebetween.

The histones used are suitably human in origin since the products of the screening method will have pharmaceutical applications, in particular in the treatment of cancer, although in view of the high degree of conservation amongst the histones, others and particularly bovine histones may be used in the screening method.

The interaction detected is suitably one in which the moiety binds to histone, and particularly H3, with an affinity which is sufficiently high so to prevent it transferring to DNA templates, and so prevent nucleosome or chromatin production in vivo. Whether the affinity of binding is sufficiently high in any particular case may be the subject of secondary screening. However, suitably the screening method employed is one in which will allow moieties with the highest affinities to be selected. Suitable screening methods would be well understood in the art.

For example, the moieties may be labelled, for example using visible labels such as radio or fluorescent labels, or immunological techniques, for example by attaching the moiety to an antibody such as horse radish peroxidase, which may be visualized using an ELISA technique. The labeled moieties are contacted with histone target which is immobilized, for example on a column or on a gel. If the moiety binds to the histone, it will be detectable by visualization of the label. The intensity of the signal will provide at least a preliminary indication of the affinity of the binding.

Preferably however, the method used is one which is amenable for high throughput screening. For example, moieties under test may be immobilized on a support, for instance in an array or within wells on a plate. They are then contacted with histone in solution, and unbound material eluted. Bound histone is then detected for example using an ELISA method. Preferably, the histone itself is labeled with a visible label so that the bound material can be readily detected.

Finally, the applicants have found that high affinity binding may cause precipitation of histones from solution. Thus, another possible screening method comprises contacting a moiety under test with a solution of histone, and detecting precipitate.

In one embodiment, aptomers may be prepared and utilized as the test moieties in the screening procedure. Once the optimum shape of the binding moiety identified in this way, if necessary a compound which mimics this shape may be prepared.

Suitable secondary screening methods will involve determining whether by binding to histones, the moiety is able to modify nucleosome formation. This can be tested using methods illustrated hereinafter. In particular, a secondary screening step will involve the attempted formation of nucleosomes from histones and DNA, which is preferably labeled, in the presence of the moiety. This can be carried out for example using methods described below, which include the “salt jump” method.

Thus in a particular embodiment, the method of the invention will further comprise a step of contacting a moiety identified by its ability to bind to a histone, as described above, with DNA and histone under conditions in which nucleosomes which form, and detecting the formation of said nucleosomes. If the moiety is able to inhibit nucleosome formation or assembly, at least at some concentrations, then it may be selected for further evaluation.

Suitable conditions are illustrated hereinafter in the experiments carried out to determine the effects of HAMLET on nucleosome assembly.

Alternatively or additionally, the moieties may be further screened to determine whether they form insoluble complexes in cell nuclei as HAMLET does in tumour cells. This can be achieved by labeling the moiety and then incubating it with the nuclear fraction of a cell extract. Salt extraction procedures, for example as described hereinafter can then be applied to solubilise nuclear material. Whether the moiety remains within the insoluble fraction or is solubilised can be determined by monitoring the amount of label material retained with the soluble and insoluble fractions.

Moieties, which may be chemical compounds, as well as proteins or peptides, identified using the screening methods defined above, for use in killing cells, form a further aspect of the invention. They may be obtained from compound collections or libraries, or may be synthesized for the screening process.

Particular novel moieties which form a further aspect of the invention are those which are capable of killing cells, and which comprising a histone binding agent, and at least one of a translocation peptide, a nuclear targeting entity, or a cell specific targeting reagent. The histone binding agent may be an agent identified using a screening as described above. Any cell specific targeting reagent is suitably specific for a tumour or undifferentiated cell, and is suitably a tumour cell specific antibody or a binding fragment thereof.

Nucleic acids encoding such moieties are also novel and may form a further aspect of the invention. These nucleic acids may be used in the transformation of vectors used to result in in vivo expression of the moiety in the cell. In this particular case, a sequence encoding a translocation peptide may not be required.

In a particular embodiment, the moiety will comprise a histone binding agent, a translocation peptide, a nuclear targeting entity, and a cell specific targeting reagent.

In particular, these compounds will find application in the treatment of conditions such as cancer or as antibacterial agents, and these methods form yet a further aspect of the invention. Pharmaceutical compositions containing these compounds, where the compounds are combined with a pharmaceutically acceptable carrier, in appropriate dosage units, in accordance with the standard practice in the art, form yet another aspect of the invention.

The affinity of HAMLET for histones was demonstrated using several different techniques as illustrated hereinafter. Interacting proteins in nuclear extracts were localized by HAMLET overlay and identified as histones. Purified histones in native, folded conformation were shown to bind HAMLET immobilized on Sepharose and surprisingly, also to form macroscopically visible precipitates with HAMLET in solution. H3 and H4 were precipitated with high efficiency and the H2A and H2B proteins were also precipitated, but with lower efficiency. BIAcore experiments confirmed the affinity of HAMLET for native core histone octamers. These results suggest that H3 is the primary targets for HAMLET in cell nuclei and suggest that this interaction is important for the effect of HAMLET in tumor cells.

HAMLET was shown to interact with intact nucleosomes, and at low concentrations, HAMLET triggered the assembly of free histones onto DNA fragments at physiological ionic strength. As HAMLET did not bind DNA, this effect must be mediated through the histones. It appears therefore that assembly may be enhanced by the high affinity binding to H3 and H4, which may help package the histones onto naked DNA. It suggests that at low concentrations, HAMLET perturbs the aggregated DNA-histone complexes to improve the conditions for nucleosome formation.

At higher concentrations, HAMLET disrupted nucleosome assembly, as shown by the disappearance of the nucleosome band. Also, when HAMLET was mixed with histones prior to the addition of DNA, no nucleosomes were formed, suggesting that the high affinity for HAMLET prevents the histones from binding to DNA. This was in contrast to a known nucleosome assembly protein, (NAP-1) (Ishimi et al., (1987) Eur J. Biochem. 162, 19-24), which induced nucleosome assembly under the same conditions. As a consequence, HAMLET was able to also release histones from aggregates with DNA that remained in the wells without entering the gel.

It is suggested that HAMLET does not act as classical nucleosome assembly protein, even thought it enhances nucleosome assembly at low concentrations. It seems possible that HAMLET freezes the chromatin due to the affinity for the histones, and thus prevents the cell from transcription, replication and recombination. Kinetic studies have indeed shown that DNA and RNA synthesis are abrogated within minutes after HAMLET treatment.

Other histone binding proteins have been identified and shown to act as chaperones during chromatin assembly and remodeling (Ito et al. (1997) Genes Cells 2, 593-600). These proteins depend on the reversibility of histone binding, as the protein must deliver the histones from the site of synthesis in the cytoplasm to the nucleus, and the chaperones are not to be part of the mature nucleosome complex. However, HAMLET does not appear to share homology with this molecules.

These findings suggest a possible molecular explanation for the accumulation of HAMLET in nuclei of tumor cells. In the nucleus, HAMLET may prevent chromatin assembly and interfere with intact chromatin, thus causing irreversible damage and cell death. This cell death should occur independently of the classical apoptotic machinery of the cell. In the cytoplasm, HAMLET may being histones and by preventing their transport to the nucleus, HUT would inhibit chromatin assembly, block replication, transcription and recombination and lead to cell death.

This new mechanism for DNA injury and cell death may also explain why HAMLET can trigger cell death in so many different tumour cell types. There are many examples of tumors escaping classical apoptotic signals, for example through p53 mutations and over-expression of the bcl-2 protein family (Johnstone et al, 2002, Cell, 2, 593-600. However, the applicants have found that HAMLET induces cell death regardless of p53 genotype and bcl-2 expression. The cell death mechanisms stimulated by HAMLET therefore appear more fundamental, attacking the chromatin assembly machinery, ultimately needed for the replication of the genome. This mechanism does not exclude the involvement of the classical apoptotic machinery with activation of the mitochondria and caspases in the activation and/or execution of cell death (Kohler et al. 1999, Exp. Cell Res. 249, 260-268).

The invention will now be particularly described by way of example, with reference to the accompanying drawings in which

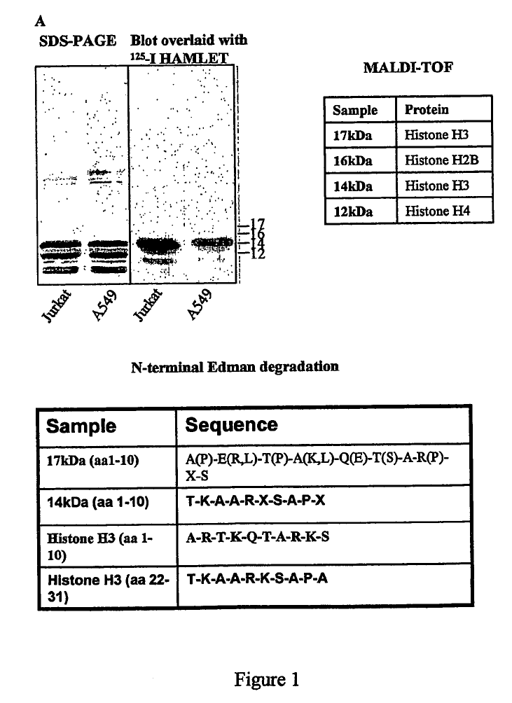

FIG. 1. HAMLET interacts with histones in nuclear extracts. (A) Nuclei were isolated from Jurkat and A549 cell lysates, digested with micrococcal nuclease to solubilize the chromatin and lysed with Triton X-100. The nuclear extracts were run on polyacrylamide SDS gels, blotted to PVDF membranes and exposed to ¹²⁵I-labelled HAMLET or monoclonal anti-H3 antibodies. Membrane blots overlaid with radiolabelled HAMLET showed binding to four bands, identified as histones by MALDI-TOF analysis or N-terminal sequencing.

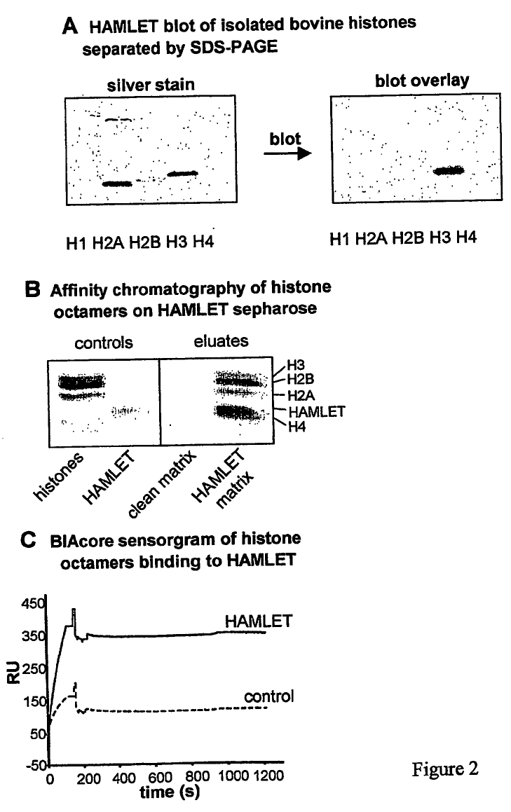

FIG. 2. Evidence for interaction with purified histones. (A) Purified bovine histones H1, H2A, H2B, H3 and H4 were subjected to SDS-PAGE and the gel was silver stained. A parallel gel was blotted to PVDF membrane and incubated with ¹²⁵I-labelled HAMLET. HAMLET bound strongly to histone H3 and H4 and weakly to H2B. No binding to histones H2A and H1 was detected. (B) Affinity chromatography of histones on HAMLET-sepharose. Histones H2A, H2B, H3 and H4 were eluted from the HAMLET-sepharose in approximately equal amounts. There was no binding to clean sepharose matrix. (C) Histone octamer binding to the BIAcore sensor chip. Biotinylated HAMLET was coupled to a streptavidin sensor chip. Native core histones octamers (100 μg/ml) were flowed over the chip and the binding was measured in resonance units (solid line) and compared to an uncoated surface (dashed line).

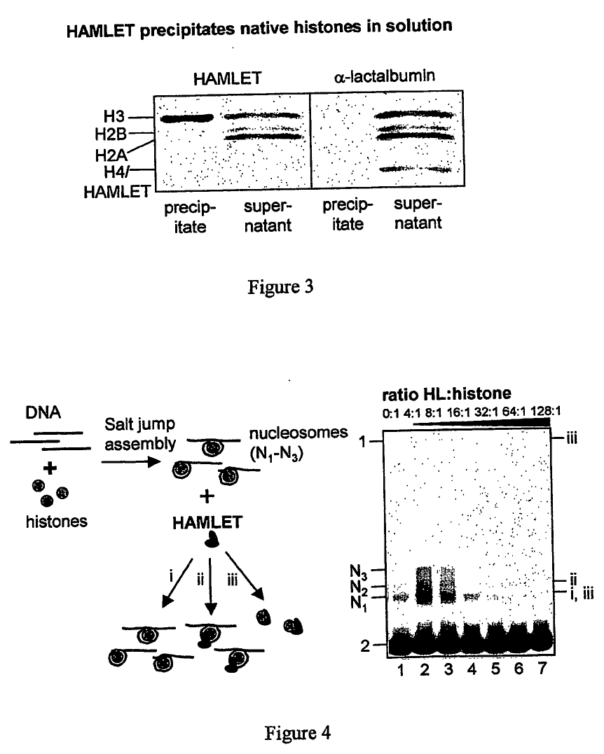

FIG. 3. HAMLET precipitates core histones from solution. Native core histones were mixed with HAMLET or α-lactalbumin. The precipitates and supernatants were subjected to SDS-PAGE with silver staining. HAMLET precipitated histones H3 and H4, but α-lactalbumin did not form precipitates with any of the histones.

FIG. 4. HAMLET binds to chromatin and influences chromatin assembly. Chromatin was assembled from isolated native core histones and radiolabelled 256 bp DNA fragments according to the salt jump method (lane 1). The products were analyzed by PAGE followed by autoradiography, and shown to consist of a mixture of unspecific histone-DNA fragments (band 3). Depending on the position of the histone octamer on the DNA fragment, variant mononucleosome species are formed (bands 2a, b and c). The effect of HAMLET on preassembled nucleosomes is shown (lanes 2-7). Increasing concentrations of HAMLET were added to the chromatin mixture and the products were analyzed. Lanes 2-3: HAMLET bound to free or aggregated histones and promoted their assembly onto DNA fragments, leading to an increase in the nucleosome band (i) with a peak at 4:1. Lanes 5-7: HAMLET associated with the nucleosomes to create a new band of lower electrophoretic mobility. (ii). Lanes 4-7: HAMLET bound to histones in nucleosomes and removed them from the DNA, resulting in a decrease in the nucleosome band (iii). HAMLET also dissolved the unspecific histone-DNA aggregates (iii).

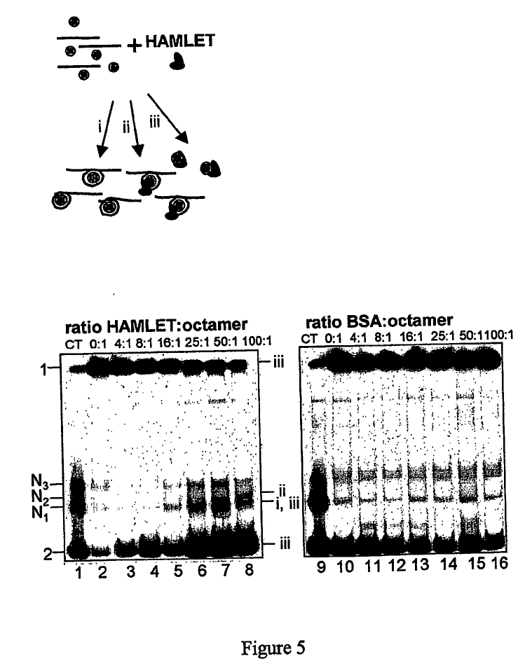

FIG. 5. HAMLET binds to chromatin and influences chromatin assembly. Histones and radio-labeled DNA fragments were mixed in 0.2M NaCl, and spontaneous nucleosome assembly was studied without the salt jump step. The starting mixture (lanes 2 and 10) contained some mono-nucleosomes, but mainly unspecific aggregates (band 1). Nucleosome assembly after salt jump is shown as a control in lanes 1 and 9. The effect of HAMLET on spontaneous nucleosome assembly is shown (lanes 2-8). Lanes 5-7: HAMLET bound to free or aggregated histones and promoted their assembly onto DNA fragments, leading to an increase in the nucleosome band (i). Lane 6-8: HAMLET associated with nucleosomes to create a new band of lower electrophoretic mobility (ii). Lane 8: HAMLET bound to histones in nucleosomes and removed them from the DNA, resulting in a decrease in the nucleosome band (iii). Lanes 3-8: HAMLET also dissolved the unspecific histone-DNA aggregates (iii). BSA at equimolar amounts showed none of these effects.

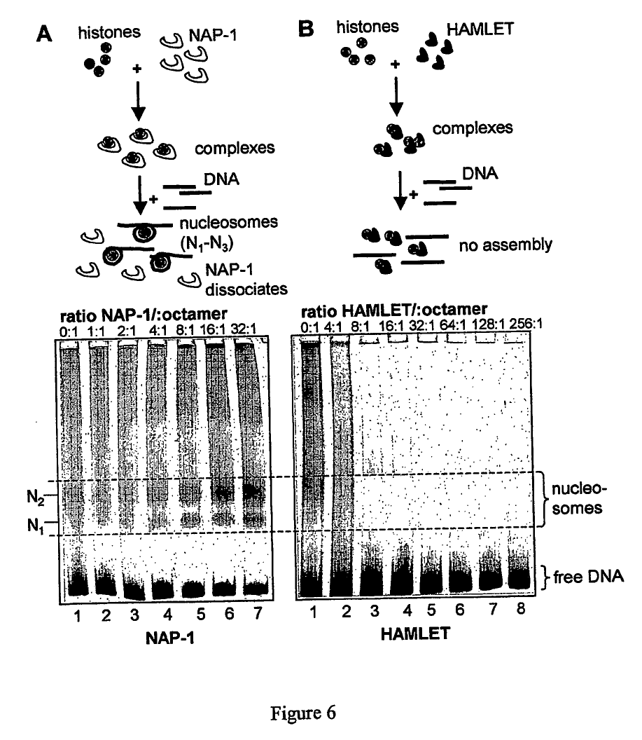

FIG. 6. HAMLET prevents chromatin assembly when premixed with histones. Core histones were preincubated with HAMLET or NAP-1 followed by the addition of DNA fragments and the assembled products were analyzed by PAGE. The histone-DNA mixture formed unspecific aggregates (lane 1 in A and B). Addition of NAP-1 (A, lanes 2-7) resulted in a concentration dependent nucleosome assembly (bands 1 and 2). After addition of HAMLET (B, lanes 2-8), no nucleosomes were formed but the unspecific aggregates were dissolved.

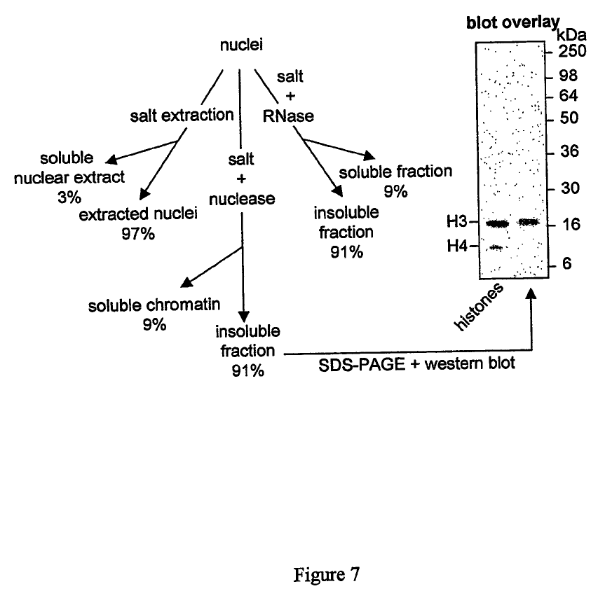

FIG. 7. HAMLET-histone interactions in vivo. Nuclei from HeLa cells treated with ¹²⁵I-HAMLET for 6 h were subfractionated, either by salt extraction, solubilization with RNase I or solubilization of chromatin with micrococcus nuclease, both treatments in the presence of salt to release soluble proteins. Soluble and insoluble fractions after each treatment were separated by centrifugation and the radioactivity associated with each fraction was measured and are shown as percentages of total nucleus-associated radioactivity. The remaining insoluble material after nuclease cleavage, containing the majority of the HAMLET taken up by the cells, was solubilized and subjected to a blot overlay assay with ¹²⁵I-HAMLET with core histones as a control.

### EXAMPLE 1

Materials and Methods

Purification of α-lactalbumin and Conversion to HAMLET.

HAMLET is a folding variant of human α-lactalbumin stabilized by a fatty acid cofactor, C18:1. Native α-lactalbumin was purified from human milk and converted to the active form (HAMLET) as previously described (Svensson, 2000 Proc. Natl. Acad. Sci USA, 97,4221-6].

Protein labeling. HAMLET was labeled with ¹²⁵I according to the lactoperoxidase method as previously described (H\dot{a}kansson, 1999 Exp Cell Res, 246, 451-60]. HAMLET was biotinylated according to the manufacturer's instructions (Boehringer Mannheim GmbH, Germany).

Cell culture and bioassays of cell death. A549 (ATCC, CLL 185), Jurkat (European Cell Culture Collection, no. 88042803) and the primary HRTEC (Human Renal Tubular Epithelial) cells were cultured as described [H\dot{a}kansson, 1995 Proc. Nalt. Acad Sci USA, 92, 8064-8]. The cells were treated with HAMLET and cell viability and DNA fragmentation were determined after 6 or 24 h of incubation, as described [H\dot{a}kansson, 1995 supra., Svennsson 2000, supra.]. Cellular localization of HAMLET. For real-time subcellular localization studies, A549 or HRTEC cells were incubated with Alexa Fluor labeled HAMLET (7 μM) under cell culture conditions described above and analyzed in a Bio-Rad 1024 laser scanning confocal equipment (Bio-Rad Laboratories, Hemel-Hempstead, UK) attached to a Nikon Eclipse 800 microscope (Nikon, Japan).

Gel Electrophoresis.

The histones and nuclear extracts were separated on tris-glycine polyacrylamide gels (15 or 16%). Bi-Tris gels (4-20%) were used to separate bovine histone-HAMLET precipitates on a Novex NuPage Mini Cell II (Novex, San Diego, Calif.). Protein bands were visualized with Coomassie blue or silver staining [Wray et al., 1981 Anal Biochem 118, 197-203]. Chromatin samples were electrophoresed at room temperature in 4% polyacrylamide (acrylamide:bisacrylamide, 29:1, w/w) either in 10 mM Tris-HCl(pH 7.5) and 1 mM EDTA (TE buffer) (Hamiche et al. 1998, J. Biol Chem., 273, 9261-9) or in 25 mM Tris, 190 mM glycine buffer with 1 mM EDTA (pH 8.3) (TEG buffer) (Hamiche et al., (1996) Proc. Natl. Acad. Sci USA 93, 7588-93. Radioactivity was quantified using a phosporimager (Fuji PC-Bas). Alternatively, the chromatin was stained with SYBR green and detected with a FluorImager (Molecular Dynamics).

Nuclear extracts. Jurkat and A549 cells were harvested, washed twice in PBS and suspended in homogenization buffer (10 mM Tris-HCl, 5 mM MgCl₂ and 2 mM CaCl₂ for Jurkat cells and 5 mM EDTA, pH 8 for A549 cells, both buffers containing 10 μg/ml leupeptin, 20 μg/ml antipain and PMSF) on ice for 15 min. The cells were homogenized (Dounce homogenizer, pestle size 411) and sucrose was added to a final concentration of 0.25 M. Nuclei were collected by centrifugation at 1000 g for 10 min, digested with micrococcus nuclease (Morales et al. (2000) Mol Cell Biol 20, 7230-7, Sigma, St Louis, Mo., USA) for 10 min at 37° C. The reaction was stopped by the addition of 5 mM EDTA and the digested nuclei were collected by centrifugation at 100 g and lysed in 1 mM EDTA. Protein concentrations were measured with BioRad DC protein assay kit.

Histones. Individually purified bovine histones H1, H2A, H2B, H3 and H4 were purchased from Roche Diagnostics (Bromma, Sweden). Native folded histones were obtained from duck erythrocyte nuclei (Simon et al., 1979 Nucleic Acids Res. 6, 689-96]. Drosophila melanogaster histones were expressed in E. coli, purified and assembled into octamers (Luger, K., et al. (1997) J. Mol. Biol. 272, 301-311). The fold and functional integrity of the histones were confirmed by nucleosome assembly on DNA (see below).

DNA. A 256-bp fragment containing a sea urchin 5S RNA gene (Simpson & Stafford (1983) Proc. Natl. Acad. Sci USA, 80, 51-5) was gel-purified from an EcoR1 digest (New England Biolabs Inc, Beverly, Mass.) of plasmid pLV405-10 (ref). The DNA was end-labeled with [γ-³²P] ATP (Amersham Pharmacia biotech, UK).

Chromatin assembly. To generate nucleoprotein particles, histone octamers were assembled on linear DNA according to the “salt jump” method (Stein, 1979 J. Mol. Biol. 130, 103-34]. ³²P-labelled 256 bp fragments and carrier DNA (supercoiled plasmid DNA, final DNA concentration 200 μg/ml) were mixed with histones (histone:DNA weight ratio 0.4-0.6) in 2 M NaCl, 10 mM Tris-HCl (pH 7.5). The mixture was incubated for 10 min at 37° C., diluted to 0.5 M NaCl, incubated at the same temperature for 30 min, and finally dialyzed at 4° C. against TE buffer for 2 h. For experiments involving SYBR green staining of the gels, the carrier plasmid DNA was substituted with nonradioactive 256 bp DNA fragments. The chromatin was stored at 4° C.

Overlay. Nuclear extracts and commercial histones were separated by PAGE and blotted to a PVDF (poly (vinylidene difluoride)) membrane. After blocking with solutions Sat 1 (ethanolamine 6.1 g/l, glycine 9 g/l, polyvinylpyrrolidone 10 g/l, methanol 25%) and Sat 2 (ethanolamine 6.1 g/l, glycine 9 g/l, Tween-20 1.25 g/l, gelatina hydrolysate 5 g/l, methanol 25%) for 15 min each, the membrane was washed 3×15 min with PBS-T (PBS, 0.05% Tween-20), incubated with ¹²⁵I-labelled HAMLET in PBS overnight, washed in PBS-T 6×15 min and dried. Bound HAMLET was detected using a STORM 840 phosphor imager (Molecular Dynamics, Inc.).

Protein sequencing and identification. Nuclear extracts were blotted onto PVDF membranes, stained with Coomassie blue and the bands to be sequenced were excised and subjected to N-terminal amino acid sequencing by Edman degradation in an Applied Biosystems model 477 A peptide sequencer. Sequences were compared to proteins of the Swiss Prot database with the PatScan software (http://www-unix.mcs.anl.gov/compbio/PatScan/HTML/patscan.html).

Mass spectrometry. The 12, 14, 16 and 17 kDa bands from the nuclear extract was excised from the gel and prepared for mass spectrometry analysis with a Bruker Scout 384 Reflex III matrix-assisted laser desorption/ionization time-of-flight (MALDI-TOF) mass spectrometer. (Lorenzo, 2001 J. Biol Chem. 276, 12201-11].

Affinity chromatography. HAMLET was immobilized on CNBr activated sepharose 4B™ (Amersham Pharmacia BiotechAB, Uppsala, Sweden) according to manufacturer's instructions. The gel was mixed with core histones in TE buffer with 0.2M NaCl for 2 h at room temperature, washed, and bound material was eluted by boiling in SDS electrophoresis buffer. The eluted material was analyzed by PAGE.

Surface plasmon resonance. Biotinylated HAMLET was immobilized on a SA sensor chip in a BIAcore X facility (BIAcore AB, Uppsala Sweden). Histone octamers were applied in serial dilutions. A flow rate of 5 μl/min was used during the immobilization and 20 μl/min during the analysis. PBS with 0.05% Tween-20 and 1M NaCl was used as flow buffer. The surface was regenerated with 10 or 20 mM HCl. The data were analyzed with the BIAevaluation 2.2 software.

Precipitation of histones with HAMLET. Histones (Roche, 25 μg) and HAMLET (25 μg) were mixed in PBS. Precipitates were harvested and the supernatants and the precipitates were subjected to SDS-PAGGE on 4-12% Bis-Tris gels (Novex). Pure histone samples were run for reference.

For detection of HAMLET in the precipitates, ¹²⁵I-labelled HAMLET was added to the reaction and the gel was analysed in the phosphor screen scanner.

Total core histones (native from cells, 2.5 μg) were mixed with HAMLET (2.5 μg) in TE buffer containing 0.2M NaCl. The mixture was left at room temperature for 5 min and the precipitate was collected centrifugation at 7000 g for 2 min. The precipitate and supernatant were analyzed by 15% Laemmli PAGE.

Results

Histones are targets for HAMLET in nuclear extracts. It has previously been shown that that HAMLET kills a wide range of tumor cells. Furthermore, nuclear localization has been demonstrated (WO 99/27967) by laser scanning confocal microscopy. It appeared from these localization studies that HAMLET interacts with distinct nuclear components in tumor cells. These were identified using nuclear extracts from A549 and Jurkat cells in the overlay assay described above with ¹²⁵T-labelled HAMLET. HAMLET was shown to bind with high intensity to four bands with approximate molecular weights of 12, 14, 16 and 17 kDa (FIG. 1A).

The 17, 16, 14 and 12 kDa bands were identified as histones H3, H2B, H3 (a proteolysed form) and H4 by MALDI-TOF. The 17 kDa band showed N-terminal homology to histone H3 and the identity was verified by immunoblot, using monoclonal anti-H3 antibodies (not shown). The 14 kDa band showed sequence homology with H3 but lacked the first 21 amino acids of the N-terminal tail (FIG. 1). This form of H3 corresponds to a fragment obtained after proteolytic degradation, and as a consequence, the band was not recognized by the anti-H3 antibody with is directed to the tail region. Hamlet, in contrast, bound to this form of H3, suggesting that the interaction is independent of the histone tail.

Interaction of HAMLET with purified histones. These results suggested that HAMLET binds histones in tumor cells. To further understand these interactions, purified histone proteins were used. First, the affinity of HAMLET for the different histone proteins was studied using commercially available purified bovine histones H1, H2A, H2B, H3 and H4. After separation by SDS-PAGE, blots were incubated with radiolabelled HAMLET (FIG. 2A). Weak binding to H2B, intermediate binding to H4 and a high affinity binding to H3 were observed. HAMLET did not bind to histone H1 or H2A in the overlay assay.

The affinity of HAMLET for histones was further examined by affinity chromatography. HAMLET was immobilized on CnBr-activated sepharose and a mixture of the core histones (H2A, H2B, H3 and H4) was applied. After washing to remove unbound protein, bound histones were eluted from the matrix with SDS loading buffer. The eluate contained the four histones in approproximately equal amounts (FIG. 2B).

The affinity of HAMLET for histones was confirmed by surface plasmon resonance, using HAMLET coated BIA core sensor chips. Two different histone preparations were used. The bovine histone H3 preparation showed virtually irreversible binding with no evidence of dissociation during the experimental period (data not shown). Furthermore, H3 could not be forcibly eluted from the chip using detergents, salt, acid etc. Subsequently, native core histone octamers were used and they bound with high affinity to the HAMLET surface as illustrated in FIG. 2C.

These results indicate that HAMLET binds isolated core histones with high affinity.

HAMLET precipitates histones from solution. Each of the purified histones H2A, H2B, H3 and H4 was mixed with HAMLET in solution. Immediately, the solution became opalescent and with time a white precipitate accumulated at the bottom of the test tube.

The precipitate was analyzed by SDS-PAGE and was shown to mainly contain histones H3 and H4 and minor amounts of H2A and H2B (FIG. 3). HAMLET was also present in the precipitates (not shown). Native α-lactalbumin was used as a control and did not form precipitates with the histones (FIG. 3) and the histones did not precipitate in the absence of HAMLET (not shown).

HAMLET interacts with chromatin in vitro. The results of the binding and precipitation assays suggested that HAMLET may interact directly with soluble histones and/or nucleosomes in vivo, and that HAMLET influences the chromatin structure in tumour cells. This hypothesis was first tested in vitro using intact nucleosomes.

Nucleosomes are formed in vitro from histones and DNA by the “salt jump” method [Stein, 1979 supra.]. Equimolar amounts of core histones are mixed with labeled 256 bp DNA fragments and a carrier plasmid in 2M NaCl and nucleosome assembly is induced by a rapid drop in molarity to 0.5M NaCl. Under these conditions the H3/H4 tetramers bind DNA and combine with the H2A/H2B dimers to form complete nucleosomes (FIG. 4).

Preformed nucleosomes obtained in this way, were mixed with HAMLET and examined by native polyacrylamide gel electrophoresis (FIG. 4). Two effects were observed. At low concentrations, HAMLET increased the nucleosome band, suggesting further nucleosome assembly from residual DNA and histones in the mixture. At higher concentrations, HAMLET caused a decrease in the mononucleosome band accompanied by the appearance of a new band, which may consist of a complex of HAMLET and nucleosomes.

HAMLET was added to the histone-DNA mixtures, and chromatin assembly was quantified without the salt jump step (FIG. 5). HAMLET was shown to cause a concentration dependent nucleosome assembly, with a maximum at 50 molecules per histone octamer. At higher concentrations of HAMLET, the mono-nucleosome band decreased, and the new band appeared (FIG. 4, band ii). In parallel, a concentration related increase in the free DNA band was observed, consistent with the ability of HAMLET to bind the histones and prevent their unspecific association with DNA. BSA was used as a control, and had no effect on nucleosome assembly. Control experiments were performed to investigate if HAMLET binds DNA. Free 256 bp DNA was mixed with HAMLET and analyzed on native polyacrylamide gel. No binding was detected (not shown).

It appears therefore that HAMLET binds to intact nucleosomes due to a direct interaction with the histones and that the binding can modify nucleosome assembly. Furthermore, the results suggest that HAMLET can displace histones from chromatin at high concentrations.

Effects of HAMLET on nucleosome assembly. The results suggested that HAMLET influences nucleosome assembly. To address this question, nucleosome assembly without the “salt jump” step was examined. Nucleosome assembly was quantified as the intensity of the mononucleosome band (FIG. 5).

HAMLET was shown to cause a dramatic, concentration dependent increase in nucleosome assembly, with a maximum at 1:50 molecules per nucleosome. It appears therefore that HAMLET enhances nucleosome assembly.

At higher concentrations of HAMLET, the mononucleosome band decreased, and a new band appeared (FIG. 5 band ii). This may represent a complex of nucleosomes and HAMLET. In parallel, the large histone-DNA aggregates were consumed and an increase in the free DNA band was observed, suggesting that HAMLET dissolved the aggregates and released DNA.

These effects were specific for HAMLET, as BSA had not effect on nucleosome assembly (FIG. 5).

It appears that HAMLET enhances the assembly of chromatin from histones and DNA and forms complexes with the chromatin. Finally, the results suggest that HAMLET can displace histones from chromatin at high concentrations.

HAMLET is not a chromatin assembly protein. A chromatin assembly protein is able to deliver histones to DNA and to enhance the formation of nucleosomes. In our model, NAP-1 (Nucloesome assembly protein 1) was used as a positive control. NAP-1 was mixed with histones, DNA fragments were added to the mixture and the assembled nucleosomes were detected by native PAGE, with a tris-glycin buffer system. NAP-1 was shown to induce nucleosome assembly in a concentration dependent manner (FIG. 6A).

The same assay system was used to test if HAMLET could act as a chromatin assembly protein, but HAMLET did not induce nucleosome assembly (FIG. 6B). In contrast, HAMLET appeared to keep the histones from binding to the labeled DNA.

These results confirmed that HAMLET binds with very high affinity to free histones and thereby inhibits nucleosome assembly if allowed to interact with free histones rather than assembled nucleosomes.

HAMLET forms an insoluble, histone containing complex in tumor cell nuclei. Nuclei were purified from ¹²⁵I-HAMLET treated HeLa cells, and the HAMLET-containing nuclear fraction was purified (FIG. 7). After measurements of total nucleus-associated radioactivity, nuclei were extracted with 0.3M KCl to release soluble nuclear proteins. Over 95% of the radioactivity remained in the insoluble nuclear fraction. The nuclei were further solubilized by treatment with RNaseI and 0.25M NaCl. Labeled HAMLET (91%) remained in the insoluble fraction. Finally, the chromatin was solubilized by micrococcus nuclease cleavage in the presence of 0.25M KCl. This treatment released chromatin from the nuclei (not shown), but the majority of HAMLET (91%) and some chromatin (not shown) remained in the insoluble fraction. This fraction was forced into solution at 95° C. in Laemmli buffer and analyzed by PAGE. Molecular species interacting with HAMLET were identified by blotting with the radio-labeled protein. HAMLET recognized histones H3 and H4 on the blot (FIG. 7).

1. A method for killing cells, said method comprising introducing into the nucleus of the cell, a moiety (other than HAMLET) which interacts with histone in a manner which is interaction is independent of the histone tail and which inhibits histone activity.

2. A method according to claim 1 wherein the moiety specifically binds histone.

3. A method according to claim 1 wherein the moiety binds H2, H3 and/or H4 histone.

4. A method according to claim 3 wherein the moiety binds H3 and/or H4 histone.

5. A method according to claim 4 wherein the moiety binds the H4 histone.

6. A method according to claim 4 wherein the moiety binds the H3 histone.

7. A method according to claim 1 wherein the moiety comprises a histone specific antibody or a binding fragment thereof.

8. A method according to claim 1 wherein the moiety acts at the DNA or RNA level to inhibit expression of functional histone.

9. A method according to claim 8 wherein the moiety comprises a sense or antisense RNA construct.

10. A method according to claim 1 wherein the moiety further comprises a carrier protein or peptide, which is able to translocate the moiety into the cell,

11. A method according to claim 1 wherein the moiety further comprises a targeting entity which locates with nucleosomes within the cell.

12. A method according to claim 11 wherein the said targeting entity is a nuclear targeting signal peptide.

13. A method according to claim 1 wherein the moiety further comprises a cell-specific targeting reagent which is specific for tumour or undifferentiated cells.

14. A method according to claim 1 wherein the cell is contacted with a vector which includes a nucleotide sequence which encodes the moiety, and is capable of expressing said sequence in-vivo in the cell.

15. A method of screening for moieties which may kill cells, said method comprises contacting a moiety under test with a histone which lacks a histone tail, and detecting an interaction therebetween.

16. A method according to claim 15 wherein the histone is H3 or H4.

17. A method according to claim 15 wherein the histone is human or bovine.

18. A method according to claim 15 wherein moieties under test are labelled, and then contacted with immobilized histone target.

19. A method according to claim 15 wherein moieties under test are immobilized on a support, contacted with histone in solution, unbound material removed, and bound histone is detected.

20. A method according to claim 19 wherein the histone is labeled.

21. A method according to claim 15 which comprises contacting a moiety under test with a solution of histone, and detecting precipitate.

22. A method according to claim 15 which further comprises a step of contacting a moiety identified using a method according to any one of claims 15 to 21, with DNA and histone under conditions in which nucleosomes which form, and detecting the formation of said nucleosomes.

23. A method according to claim 15 which further comprises the step of determining whether the moiety forms insoluble complexes in cell nuclei.

24. A moiety identified using a method according to claim 15 for use in killing cells.

25. A moiety capable of killing cells, said moiety comprising a histone binding agent, and at least one of a translocation peptide, a nuclear targeting entity, or a cell specific targeting reagent.

26. A moiety capable of killing cells, said moiety comprising a histone binding agent, and at least one of a translocation peptide, a nuclear targeting entity, or a cell specific targeting reagent, wherein the histone binding agent is an agent identified using a method according to claim 15.

27. A nucleic acid which encodes a moiety according to claim 24, where said moiety is a polypeptide.

28. A pharmaceutical composition comprising a moiety according to claim 24.

29. The use of a moiety according to claim 21 in the preparation of a medicament for use in the treatment of cancer.

30. The use of a moiety according to claim 24 in the preparation of a medicament for use in the treatment of bacterial infections.

31. A method of killing or controlling bacteria which comprises applying to the bacteria or to the environment thereof, a moiety according to claim 24.
